(19) United States
(12) Patent Application Publication
(10) Pub. No.: US 2009/0142389 A1
(43) Pub. Date: Jun. 4, 2009

(54) HYDROGEL SHEET AND PRODUCTION METHOD THEREOF

(76) Inventors: Makoto Yamashita, Saitama (JP); Naosuke Maruyama, Niigata (JP); Kazuhisa Hayakawa, Niigata (JP)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/300,066

(22) PCT Filed: May 31, 2007
(86) PCT No.: PCT/JP2007/061500

§ 371 Date: Nov. 7, 2008

(30) Foreign Application Priority Data
May 31, 2006 (JP) 2006-151373

(51) Int. Cl. A61K 9/70

(52) U.S. Cl. 424/443

(57) ABSTRACT

Provided is a hydrogel sheet being excellent in strength and flexibility and having high water content in spite of comprising a smaller amount of polymer material; and a method for producing a hydrogel sheet which is convenient and has high productivity. More specifically, provided is a hydrogel sheet comprising (methyl vinyl ether/maleic acid) crosspolymer, low-substituted cellulose ether and water wherein the low-substituted cellulose ether has a molar substitution degree of 0.05 to 1.0 per an anhydrous glucose unit and is insoluble in water but soluble in an aqueous alkali solution.

### TECHNICAL FIELD

The present invention relates to a hydrogel sheet which can be used as a cooling sheet for relieving fever, a cosmetic sheet, an adhesive sheet such as cataplasm or the like.

### BACKGROUND OF ART

As disclosed in Japanese Patent Application Unexamined Publication Nos. 2004-231567 and 2000-72691, a cooling sheet for relieving fever conventionally comprises an adhesive layer of a hydrogel, the layer having a cooling capacity. The hydrogel for the adhesive layer is prepared by adding a crosslinking agent such as aluminum hydroxide to a polyacrylate or the like and causing a crosslinking reaction of a metal ion through a coordinate bond.

The adhesive layer of the hydrogel in which a synthetic water-soluble polymer such as polyacrylic acid has been used as a gelling agent has to have a high water content in order to enhance a cooling effect or moisturizing effect. An increase in the water content, on the contrary, leads to deterioration in strength or stability of the gel. In addition, the increase in the water content requires a longer time (one day or more) for conventional gelation using a crosslinking agent and moreover, it is difficult to adjust conditions for obtaining sufficient gel strength.

In Japanese Patent Application Unexamined Publication Nos. 2003-226633 and 2003-51800, a cooling sheet and a cosmetic sheet using a hydrogel made of a water soluble polymer material such as agar or gelatin are proposed. However, they have a problem that the gelation rate of these sheets containing various active ingredients cannot be optimized. Moreover, the gel sheet made of agar has a problem of insufficient strength.

Japanese Patent Application Unexamined Publication No. 2003-300852 discloses a sheet comprising low-substituted hydroxypropyl cellulose, which is a nonionic polymer insoluble in water but soluble in an aqueous alkali solution. Although the sheet comprising low-substituted hydroxypropyl cellulose has high sheet strength, however, it has low adhesion and flexibility. Accordingly, it has a problem that when it is applied to the skin, it easily peels off from the skin in a short time.

Japanese Patent Application Unexamined Publication No. 2005-179253 discloses a sheet comprising a mixture of the low-substituted hydroxypropyl cellulose and a water soluble cellulose ether. The sheet has a problem that when a water soluble cellulose ether is added to enhance the adhesion to the skin, the sheet strength is reduced although adhesion to the skin is improved.

### DISCLOSURE OF INVENTION

With the foregoing in view, the present invention has been completed. An object of the present invention is to provide a hydrogel sheet being excellent in strength and flexibility and having a high water content in spite of comprising a smaller amount of a polymer material, as well as a method for producing the hydrogel sheet, the method being convenient and having a high productivity.

As a result of an extensive investigation by the inventors in order to attain the above-described objects, it has been found that a hydrogel sheet being excellent in strength and flexibility and having a high water content in spite of comprising a smaller amount of a polymer material can be obtained by using (methyl vinyl ether/maleic acid) crosspolymer and low-substituted cellulose ether which is insoluble in water but soluble in an aqueous alkali solution. It has also been found that the hydrogel sheet can be obtained by a production method which is convenient and has high productivity.

More specifically, in the present invention, there is provided a hydrogel sheet comprising (methyl vinyl ether/maleic acid) crosspolymer, low-substituted cellulose ether and water, wherein the low-substituted cellulose ether has a molar substitution degree, per anhydrous glucose unit, of from 0.05 to 1.0 and is insoluble in water but soluble in an aqueous alkali solution.

In the present invention, there is also provided a method for producing a hydrogel sheet, comprising steps of dispersing (methyl vinyl ether/maleic acid) crosspolymer and low-substituted cellulose ether in water to form a dispersion wherein the low-substituted cellulose ether is insoluble in water but soluble in an aqueous alkali solution; adding an alkali solution to the dispersion and mixing them to form a gel solution; casting the gel solution onto a base plate; and rinsing the cast gel solution with an acid.

The hydrogel sheet of the present invention can form an adhesive hydrogel layer having high tensile strength and flexibility and having a high water content so that it is useful for the improvement in a cooling effect or moisturizing effect. Compared with the conventional method, the method of the present invention can produce a gel sheet more conveniently in a short time so that a cost can be reduced.

### BEST MODE FOR CARRYING OUT THE INVENTION

The present invention will hereinafter be described in detail.

The hydrogel sheet according to the present invention comprises (methyl vinyl ether/maleic acid) crosspolymer (which will hereinafter be called “PVM/MA crosspolymer”), low-substituted cellulose ether insoluble in water but soluble in an aqueous alkali solution, and water.

Although the hydrogel sheet of the present invention has both high tensile strength and flexibility, it can form a hydrogel having a high water content. A conventional method for producing a hydrogel sheet in use of a metal crosslinking agent requires heating for causing gelation and long hours to complete the gelation. On the other hand, in the present invention, the production method can be simplified because it does not require addition of a metal crosslinking agent such as aluminum hydroxide and gelation proceeds smoothly at an ordinary temperature. Moreover, the production method can be controlled easily, making it possible to reduce the production cost.

When an aqueous solution of sodium hydroxide is added to an aqueous suspension of a PVM/MA crosspolymer, a hydration reaction of acid anhydride of the PVM/MA crosspolymer takes place to form the corresponding dicarboxylic acid. As the amount of an alkali increases, the reaction rate increases. In the presence of an excess alkali, the reaction rate becomes the maximum.

When the reaction takes place in the presence of low-substituted cellulose ether which has been dispersed uniformly in water, wherein the low-substituted cellulose ether is insoluble in water but soluble in an aqueous alkali solution, a hydration reaction of acid anhydride of PVM/MA crosspolymer takes place to form the corresponding dicarboxylic acid. In addition, low-substituted cellulose ether insoluble in water but soluble in an aqueous alkali solution is dispersed in a network of the PVM/MA crosspolymer by taking an advantage of its property of dissolving in an aqueous alkali solution. The carboxyl group of the PVM/MA crosspolymer forms a crosslink with the hydroxyl group of the low-substituted cellulose ether through a hydrogen bond, whereby gelation occurs instantly.

Since the low-substituted cellulose ether to be used in the present invention which is insoluble in water but soluble in an aqueous alkali solution is a linear polymer, it has an effect of improving the strength of the gel thus formed. A hydrogel sheet consisting only of low-substituted cellulose ether insoluble in water but soluble in an aqueous alkali solution has high sheet strength but has poor flexibility. However, a hydrogel sheet comprising PVM/MA crosspolymer which is a branched polymer has its flexibility heightened so that the hydrogel sheet of the present invention has high tensile strength and high flexibility.

The PVM/MA crosspolymer is listed in The Cosmetic Ingredient Names and the List as methyl vinyl ether/maleic acid) crosspolymer, but is commonly known as PMM/MA crosspolymer or (methyl vinyl ether/maleic anhydride) crosspolymer. It is commercially available under the trade name of “STABILEZE” from ISP Japan Ltd. It can be prepared in accordance with the method as described in US Patent No. 5874510.

The PVM/MA crosspolymer is a crosslinked polymer produced by radical polymerization of methyl vinyl ether and maleic anhydride with a small amount of an end-unsaturated diene. For example, PVM/MA crosspolymer produced by radical polymerization of methyl vinyl ether and maleic anhydride with a small amount of decadiene shows characteristic absorptions at 2929, 2830, 1854, 1779, 1730, 1223, 1094 and 927 cm⁻¹. It is supplied as a white powder. When it is dispersed in water and then heated, a hydration reaction takes place to form dicarboxylic acid. This hydration reaction is accelerated markedly by an addition of an alkali at an ordinary temperature and the subsequent neutralization produces a gel having a high viscosity. The resulting gel has a viscosity, in the form of a 0.5w by weight aqueous solution, of from 40,000 to 80,000 mpa·s.

A molar ratio of methyl vinyl ether to maleic anhydride in the PVM/MA crosspolymer may be preferably from 40:60 to 60:40, especially preferably from 45:55 to 55:45.

The end-unsaturated diene compound usable for the crosslinking may include an end-unsaturated diene compound having from 6 to 18 carbon atoms. Specific examples may include 1, 5-hexadiene, 1, 7-octadiene, ethylene dimethacrylate, methacrylic anhydride and diallyl phthalate. Of these, 1,9-decadiene may be especially preferred.

The degree of crosslinking of the PVM/MA crosspolymer may be preferably from 1 to 5 mole%, especially preferably from 2 to 4 mole%. Its weight average molecular weight may be preferably 1,000,000 or greater.

The content of the PVM/MA crosspolymer in the whole hydrogel sheet may be preferably from 0.1 to 10% by weight, especially preferably from 0.4 to 5% by weight. When the content is less than 0.1% by weight, the resulting sheet may not have sufficient sheet strength. When the content is more than 10% by weight, a hydrogel sheet may not be produced because viscosity may become too high so that smooth mixing of the crosspolymer may be disturbed. Thus, there may be a case or cases wherein the hydrogel cannot be produced.

The PVM/MA crosspolymer can provide a markedly high viscosity at a low concentration. The resulting gel has low strength and is therefore fragile so that it may be difficult to be used as a gel sheet.

According to the present invention, low-substituted cellulose ether insoluble in water but soluble in an aqueous alkali solution is added in order to heighten the tensile strength of the hydrogel sheet and to form a gel in a short time at an ordinary temperature without adding a metal crosslinking agent such as aluminum hydroxide for crosslinking polyacrylate or the like.

Examples of low-substituted cellulose ether insoluble in water but soluble in an aqueous alkali solution may include low-substituted hydroxypropyl cellulose (L-HPC) (molar substitution degree of from 0.05 to 1.0), low-substituted hydroxyethyl cellulose (molar substitution degree of from 0.05 to 1.0), low-substituted carboxymethyl cellulose (L-CMC) (molar substitution degree of from 0.05 to 0.3), and low-substituted hydroxypropylmethyl cellulose (molar substitution degree of from 0.05 to 1.0). Of these, low-substituted hydroxypropyl cellulose may be preferred because of excellent solubility in an alkali.

The content of the low-substituted cellulose ether insoluble in water but soluble in an aqueous alkali solution may be preferably from 50 to 150% by weight relative to an amount of the PVM/MA crosspolymer. When the content is less than 50% by weight, the resulting hydrogel sheet may have insufficient strength. When the content is more than 150% by weight, the resulting gel sheet may have reduced flexibility.

The low-substituted cellulose ether insoluble in water but soluble in an aqueous alkali solution will be described in detail. Cellulose is generally insoluble in water. When the hydrogen atom of the hydroxyl group of the glucose ring of the cellulose is substituted with an alkyl or hydroxyalkyl group, the substituted cellulose acquire water solubility, depending on the substitution degree. Low-substituted cellulose having a low substitution degree exhibits no solubility in water but very frequently becomes soluble in an alkali solution. In most cases, when the low-substituted cellulose is dispersed in water, it is partially swelled with water. Cellulose having a high molar substitution degree becomes soluble in water, while losing solubility in an alkali.

The low-substituted cellulose ether insoluble in water but soluble in an aqueous alkali solution has a molar substitution degree of from 0.05 to 1.0. When the molar substitution degree is out of the range, the cellulose ether becomes insoluble in an aqueous alkali solution so that the resulting gel sheet cannot have increased strength. The substitution degree of the low-substituted cellulose ether can be determined by isolating the substituent and then analyzing with a gas chromatograph in accordance with the description in “Low-substituted hydroxypropyl cellulose” of the Japanese Pharmacopoeia 15th Edition. The low-substituted cellulose ether insoluble in water but soluble in an aqueous alkali solution, for example, low-substituted hydroxypropyl cellulose can be produced by using the method as descried in Japanese Patent Application Unexamined Publication 51-63927/1976 (U.S. Pat. No. 4,091,205).

The hydrogel sheet of the present invention may have an elongation of preferably 100% or greater, especially preferably 150% or greater. The elongation is a percentage of elongated length relative to the original length until breakage of the sheet occurs and can be determined in accordance with Japan Industrial Standard (JIS) K 7113.

The hydrogel sheet of the present invention has a water content of preferably 80% by weight or greater, especially preferably 85% by weight or greater so that it has high tensile strength and an excellent moisturizing effect. The water content can be determined in accordance with the Loss on Drying test of General Tests of the Japanese Pharmacopoeia, 15th Edition.

The production method of the hydrogel sheet according to the present invention will next be described.

A highly viscous gel solution can be obtained in a short time at an ordinary temperature by using a method comprising steps of disposing PVM/MA crosspolymer and low-substituted cellulose ether in water to form a dispersion wherein the low-substituted cellulose is insoluble in water and soluble in an aqueous alkali solution; and adding an alkali solution to the resulting dispersion and mixing them.

It may be preferred, in order to form an aqueous dispersion of the PVM/MA crosspolymer and low-substituted cellulose ether insoluble in water but soluble in an aqueous alkali solution, to add them in amounts permitting the formation of a uniform dispersion into water.

Examples of the alkali to be used may include sodium hydroxide, potassium hydroxide, ammonium hydroxide, monoethanolamine, aminomethylpropanol, aminomethylpropanediol, tromethamine, hydroxymethyl glycinate and tetrahydroxypropyl ethylenediamine. Sodium hydroxide may be especially preferable from the standpoint of the solubility of the low-substituted cellulose ether insoluble in water but soluble in an aqueous alkali solution.

The amount of the alkali may be preferably from 10 to 140% by weight, especially preferably from 20 to 100% by weight based on the total weight of the PVM/MA crosspolymer and the low-substituted cellulose ether insoluble in water but soluble in an aqueous alkali solution. When the amount is less than 10% by weight, the hydration rate of the PVM/MA crosspolymer is too slow to dissolve, in water, the low-substituted cellulose ether insoluble in water but soluble in an aqueous alkali solution so that it may be difficult to produce a gel sheet. When the amount is more than 1400% by weight, the solubility of the PVM/MA crosspolymer may be reduced. The alkali can be added as an aqueous solution thereof or as a mixed solution of water, a water soluble solvent such as alcohol and the alkali. It may be preferable to add the alkali in the above-described amount by using a solution having an alkali concentration of from 5 to 200 by weight.

A hydrogel produced by an addition of an alkali solution may be cast on a base plate preferably after defoaming. The base plate may include, but not particularly limited to, a glass plate and polyethylene terephthalate film. The hydrogel can be cast onto the base plate in a known manner, for example, by using a doctor blade applicator.

The hydrogel produced by the addition of the alkali solution is in most cases on the alkaline side so that rinsing the hydrogel with an acid solution can result in a hydrogel ranging from a neutral pH to a weakly acidic pH. Examples of the acid to be used for this purpose may include organic acids such as oxalic acid, malic acid and citric acid, and mineral acids such as hydrochloric acid and sulfuric acid. The concentration of the acid solution used in the step of rinsing may be preferably from 3 to 500 by weight, especially preferably from 5 to 20% by weight. When the concentration is lower than the range, the neutralization effect by rinsing may not appear smoothly, while when the concentration is too high, neutralization by rinsing may not occur uniformly, which may facilitate acid hydrolysis of such a gel composition. The rinse solution may be used in an amount which allows the hydrogel sheet to be dipped and rinsed. The rinse solution may be preferably used in an amount of from about 5 to 10 times the weight of the hydrogel sheet as an economical amount for heightening the rinsing effect. After rinsing with the acid solution, rinsing with a large amount of neutral water may be further carried out.

The hydrogel sheet of the present invention can be used as a cooling sheet, a cataplasm or a cosmetic sheet. It may comprise a various active ingredient such as a drug.

Examples of the drug may include an anti-wrinkle agent such as retinol; an anti-spot agent such as cysteine; a whitening cosmetic; a moisturizing ingredient such as glycerin, hyaluronic acid, collagen, squalene, docosahexaenoic acid, eicosapentaenoic acid, saccharides, amino acids, placenta extract, sorbitol and polyethylene glycol; a softener such as olive oil, cetyl alcohol, lanolin and stearyl alcohol; blood circulation promoters such as tocopherol, an anti-inflammatory such as glycyrrhizinic acid; and a skin beautifying agent such as various Vitamin Cs. The hydrogel sheet can comprise one or more of these drugs. If necessary, a water-soluble organic solvent such as alcohol may be added.

In use for a percutaneous absorption preparation such as cataplasm, examples of a local anesthetic may include tetracaine, diethylaminoethyl parabutylaminobenzoate, oxybuprocaine, lidocaine, dibucaine and propitocaine. Examples of an analgesic antiphlogistic may include salicylic acid, sodium salicylate, methyl salicylate, aspirin, acetaminophen, ethenzamide, ibuprofen, indomethacin, ketoprofen, glycyrrhizinic acid, flufenamic acid, phenylbutazone, naproxen, oxyphenbutazone, diclofenac sodium, benzydamine, mepirizole, isothipendyl hydrochloride, bufexamac, bendazac, azulene, piroxicam and diflunisal. Examples of an anti-inflammatory steroid may include triamcinolone acetonide, dexamethasone, hydrocortisone acetate, fluocinolone acetonide, prednisolone and betamethasone valerate. Examples of an antibiotic may include penicillin, gentamicin, cefalexin, erythromycin, chloramphenicol and tetracycline.

Although no particular limitation is imposed on the content of such a drug, it may be preferably from 0.05 to 100 by weight, especially preferably from about 0.1 to 50 by weight relative to an amount of the hydrogel sheet to which the drug has not been added (before addition of the drug).

In order to heighten the antiseptic property or stability of the hydrogel sheet, a slight amount of an antiseptic may be added.

Examples of the antiseptic may include sorbic acid and sorbic acid derivatives such as potassium sorbate and sodium sorbate; imidazolidinylurea; paraoxybenzoates; parabens and derivatives thereof such as isopropylparaben, isobutylparaben and butylparaben. An amount of the antiseptic may be preferably from 0.001 to 0.5% by weight, especially 0.01 to 0.1% by weight relative to an amount of the hydrogel sheet to which the antiseptic has not been added. It is because within this range, the antiseptic can perform its function at the minimal amount.

Such a drug or antiseptic can be added to a gel by dipping the rinsed hydrogel sheet in an aqueous solution or ethanol solution of the drug or antiseptic so as to impregnate it into the gel.

Although no particular limitation is imposed on the concentration of the drug or antiseptic, from 5 to 50% by weight may be preferred because it does not increase the viscosity of the solution.

The amount of the solution in which the hydrogel sheet will be dipped is not limited insofar as it can be dipped therein and may be preferably from 5 to 10 times the weight of the hydrogel sheet. When the drug or antiseptic is stable to the alkali solution, it may be added at any ratio during mixing of the base materials for the present invention.

For heightening adhesion, it is also possible to add a water soluble polymer such as polyvinylpyrrolidone, carboxymethyl cellulose (CMC) (molar substitution degree of from 0.3 to 0.5), hydroxypropylmethyl cellulose (molar substitution degree of from 1.5 to 2.2), methyl cellulose (molar substitution degree of from 1.5 to 2.0), hydroxypropyl cellulose (HPC) (molar substitution degree of from 2.8 to 3.2) or polyvinyl alcohol during mixing of the base materials (PVM/MA crosspolymer and low-substituted hydroxypropyl cellulose (L-HPC)). An amount of the water soluble polymer is not particularly limited and may be, from the viewpoint of heightening the adhesion, preferably from 0.1 to 10% by weight, especially preferably from 1 to 5% by weight relative to an amount of the hydrogel sheet to which the water soluble polymer has not been added. It is because the water soluble polymer can exhibits its effect at the minimal amount.

The present invention will be described specifically by Examples and Comparative Examples. It should not be construed that the present invention is limited to or by them.

### EXAMPLES 1 To 7 And COMPARATIVE EXAMPLES 1 To 4

PVM/MA crosspolymer and low-substituted cellulose ether insoluble in water and soluble in an aqueous alkali solution were weighed into the amounts as shown in Table 1. After they were mixed, the resulting mixture was charged in a 200-ml beaker, followed by the addition of a predetermined amount of water thereto. The mixture was stirred for 3 minutes at 700 rpm with a screw type agitating blade having a diameter of about 40 mm which is small enough to fit in the beaker. An alkali was then added to the beaker and stirred at 100 rpm for 1 hour with the screw type agitating blade, whereby a solution was prepared. The solution was allowed to stand at room temperature for about 12 hours to remove bubbles therefrom and then 20 to 40 ml of the solution was poured onto a polyethylene terephthalate sheet of 25 μm in thickness, 10 cm in length and 15 cm in width placed on a flat plane. By a doctor blade applicator adjusted to provide a thickness of 1.5 mm, a coated surface of about 5 cm in width and 10 cm in length was prepared. An acid solution as shown in Table 1 was charged in a bat of 10 cm in length, 15 cm in width and 5 cm in depth and the polyethylene terephthalate sheet to which the solution had been applied was dipped in it. After the sheet was rinsed for 5 minutes, it was washed with pouring water for 30 minutes.

The drug as shown in Table 1 was then placed in a bat having a similar size to that of the above-described one and the polyethylene terephthalate sheet on which the rinsed hydrogel sheet had been coated was dipped in it. After it was allowed to stand for 5 minutes, it was taken out from the bat. The drug solution was wiped off from the surface with absorbent cotton and the sheet was cut into strips of 7 cm in length and about 3 cm in width.

Tensile strength of the hydrogel sheet thus obtained was measured at a stretching rate of 10 mm/min by using a tensile strength measurement mode of a rheometer manufactured by Rheotech Co., Ltd. The tensile strength of the sheet at break and elongation relative to the original length of the sheet until the breakage of the sheet were measured. The results are shown in Table 1. The water content was determined in accordance with the Loss on Drying test of General Tests of The Japanese Pharmacopoeia 15th Edition by weighing 2 g of sample sheet in a weighing bottle, drying it at 105° C. for 4 hours and calculating the water content based on the weight loss.

As shown in Table 1, the hydrogel sheet obtained in Comparative Example 1 by using only PVM/MA crosspolymer had lower tensile strength and lower elongation than those of the sheets obtained in Examples. The hydrogel sheet obtained in Comparative Example 2 by using only L-HPC had lower elongation than that of the sheets obtained in Examples. The hydrogel sheet obtained in Comparative Example 3 by using an L-HPC having a low molar substitution degree and the hydrogel sheet obtained in Comparative Example 4 by using water soluble HPC having a high molar substitution degree showed lower tensile strength and lower elongation than those of the sheets obtained in Examples. It is evident based on these results that the hydrogel sheet obtained in the invention exhibits high tensile strength and elongation in spite of a high water content.

TABLE 1

| | step of | | | | | | evaluation | | |
|---|---|---|---|---|---|---|---|---|---|
| | | dispersing | adding | | | impregnating | tensile | elon- | water |
| | mixing powders | in water | alkali | | rising | with drug | strength | gation | content |
| | (parts by weight: pbw) | (pbw) | (pbw) | | (pbw) | (pbw) | (g) | (%) | (%) |
| Example | PVM/MA crosspolymer | water | aq. 10 wt % | total | aq. 10 wt % | — | 300 | 200 | 90 |
| 1 | (3) | added | NaOH | (100) | oxalic acid | | | | |
| | L-HPC(mol. sub. deg. of 0.2) | (44) | solution | | solution | | | | |
| | (3) | | (50) | | (1000) | | | | |
| Example | PVM/MA crosspolymer | water | aq. 10 wt % | total | aq. 10 wt % | — | 350 | 150 | 89 |
| 2 | (5) | added | NaOH | (100) | oxalic acid | | | | |
| | L-HPC(mol. sub. deg. of 0.2) | (42.5) | solution | | solution | | | | |
| | (2.5) | | (50) | | (1000) | | | | |
| Example | PVM/MA crosspolymer | water | aq. 10 wt % | total | aq. 10 wt % | — | 200 | 225 | 92 |
| 3 | (3) | added | NaOH | (100) | malic acid | | | | |
| | L-HPC(mol. sub. deg. of 0.2) | (42.5) | solution | | solution | | | | |
| | (4.5) | | (50) | | (1000) | | | | |
| Example | PVM/MA crosspolymer | water | aq. 10 wt % | total | aq. 10 wt % | aq. 50 wt % | 280 | 110 | 85 |
| 4 | (3) | added | NaOH | (100) | malic acid | glycerin | | | |
| | L-HPC(mol. sub. deg. of 0.05) | (44) | solution | | solution | solution | | | |
| | (3) | | (50) | | (1000) | (500) | | | |
| Example | PVM/MA crosspolymer | water | monoethanolamine | total | aq. 5 wt % | aq. 50 wt % | 220 | 210 | 93 |
| 5 | (4) | added | (2) | (100) | hydrocloric acid | glycerin | | | |
| | L-HPC(mol. sub. deg. of 1.0) | (92) | | | solution | solution | | | |
| | (2) | | | | (1000) | (500) | | | |
| Example | PVM/MA crosspolymer | water | aq. 10 wt % | total | aq. 5 wt % | aq. 20 wt % | 290 | 101 | 83 |
| 6 | (3) | added | NaOH | (100) | hydrocloric acid | sodium | | | |
| | L-HPC(mol. sub. deg. of 0.30) | (44) | solution | | solution | salicylate | | | |
| | (3) | | (50) | | (1000) | solution | | | |
| | | | | | | (500) | | | |
| Example | PVM/MA crosspolymer | water | aq. 10 wt % | total | aq. 10 wt % | aq. 30 wt % | 280 | 105 | 84 |
| 7 | (3) | added | NaOH | (100) | malic acid | vitamin C | | | |
| | L-CMC(mol. sub. deg. of 0.28) | (44) | solution | | solution | solution | | | |
| | (3) | | (50) | | (1000) | (500) | | | |
| Comp. | PVM/MA crosspolymer | water | aq. 10 wt % | total | — | — | 50 | 21 | 89 |
| Ex. | (8) | added | NaOH | (100) | | | | | |
| 1 | | (70.4) | solution | | | | | | |
| | | | (21.6) | | | | | | |
| Comp. | L-HPC(mol. sub. deg. of 0.2) | water | aq. 10 wt % | total | aq. 10 wt % | — | 380 | 40 | 91 |
| Ex. | (6) | added | NaOH | (100) | oxalic acid | | | | |
| 2 | | (44) | solution | | solution | | | | |
| | | | (50) | | (1000) | | | | |
| Comp. | PVM/MA crosspolymer | water | aq. 10 wt % | total | aq. 10 wt % | — | 40 | 15 | 75 |
| Ex. | (3) | added | NaOH | (100) | oxalic acid | | | | |
| 3 | L-HPC(mol. sub. deg. of 0.01) | (44) | solution | | solution | | | | |
| | (3) | | (50) | | (1000) | | | | |
| Comp. | PVM/MA crosspolymer | water | aq. 10 wt % | total | — | — | 45 | 30 | 90 |
| Ex. | (3) | added | NaOH | (100) | | | | | |
| 4 | HPC (mol. sub. deg. of 3.0) | (85.9) | solution | | | | | | |
| | (3) | | (8.1) | | | | | | |

* The number in the parenthesis in the step of adding alkali means an amount of alkali contained in the solution.
The number in the parenthesis in the step of rinsing or impregnating with drug means an amount of the added solution.

1. A hydrogel sheet comprising:
(methyl vinyl ether/maleic acid) crosspolymer, low-substituted cellulose ether having a molar substitution degree of 0.05 to 1.0 per anhydrous glucose unit and being insoluble in water but soluble in an aqueous alkali solution, and
water.

2. The hydrogel sheet according to claim 1, having an elongation of 100% or greater.

3. The hydrogel sheet according to claim 1, having a water content of 80% by weight or greater.

4. The hydrogel sheet according to claim 1, wherein said low-substituted cellulose ether insoluble in water but soluble in an aqueous alkali solution is selected from the group consisting of low-substituted hydroxypropyl cellulose, low-substituted hydroxyethyl cellulose, low-substituted hydroxypropylmethyl cellulose and low-substituted carboxymethyl cellulose.

5. The hydrogel sheet according to claim 1, I4to, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer.

6. A method for producing a hydrogel sheet, comprising steps of:
dispersing (methyl vinyl ether/maleic acid) crosspolymer and low-substituted cellulose ether in water to form a dispersion wherein the low-substituted cellulose ether is insoluble in water but soluble in an aqueous alkali solution;
adding an alkali solution to the dispersion and mixing to form a gel solution;
casting the gel solution onto a base plate; and rinsing the cast gel solution with an acid solution.

7. The hydrogel sheet according to claim 2, having a water content of 80% by weight or greater.

8. The hydrogel sheet according to claim 2, wherein said low-substituted cellulose ether insoluble in water but soluble in an aqueous alkali solution is selected from the group consisting of low-substituted hydroxypropyl cellulose, low-substituted hydroxyethyl cellulose, low-substituted hydroxypropylmethyl cellulose and low-substituted carboxymethyl cellulose.

9. The hydrogel sheet according to claim 3, wherein said low-substituted cellulose ether insoluble in water but soluble in an aqueous alkali solution is selected from the group consisting of low-substituted hydroxypropyl cellulose, low-substituted hydroxyethyl cellulose, low-substituted hydroxypropylmethyl cellulose and low-substituted carboxymethyl cellulose.

10. The hydrogel sheet according to claim 7, wherein said low-substituted cellulose ether insoluble in water but soluble in an aqueous alkali solution is selected from the group consisting of low- substituted hydroxypropyl cellulose, low-substituted hydroxyethyl cellulose, low-substituted hydroxypropylmethyl cellulose and low-substituted carboxymethyl cellulose.

11. The hydrogel sheet according to claim 2, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer

12. The hydrogel sheet according to claim 3, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer.

13. The hydrogel sheet according to claim 4, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer.

14. The hydrogel sheet according to claim 7, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer.

15. The hydrogel sheet according to claim 8, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer.

16. The hydrogel sheet according to claim 9, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer.

17. The hydrogel sheet according to claim 10, wherein said (methyl vinyl ether/maleic acid) crosspolymer is comprised in an amount of from 0.1 to 10% by weight in the whole hydrogel sheet and said low-substituted cellulose ether insoluble in water but soluble in an aqueous alkali solution is comprised in an amount of from 50 to 150% by weight relative to the amount of the (methyl vinyl ether/maleic acid) crosspolymer.
